(19) United States
(12) Patent Application Publication
(10) Pub. No.: US 2024/0075255 A1
(43) Pub. Date: Mar. 7, 2024

(54) MEDICAL BALLOON ASSEMBLY, USE THEREOF, AND METHOD OF DEPLOYING A MEDICAL BALLOON ASSEMBLY

(71) Applicant: Cook Medical Technologies LLC, Bloomington, IN (US)

(72) Inventor: Palle Hansen, Granvej (DK)

(21) Appl. No.: 18/242,110

(22) Filed: Sep. 5, 2023

(30) Foreign Application Priority Data
Sep. 7, 2022 (GB) 2213049.6

(51) Int. Cl. A61M 25/10; A61M 25/00; A61M 29/02

(52) U.S. Cl. CPC A61M 25/10; A61M 25/0045; A61M 29/02

(57) ABSTRACT

An introducer assembly includes a balloon catheter, a medical balloon at the distal end of the catheter. On the outside of the balloon catheter and medical balloon is a roll-sock sheath. The sheath is in formed of two layers able to slide over one other extending towards the proximal end of the assembly. The roll-sock sheath extends to a distal fold line which, before employment, extends over at least a portion of dilator tip and covers completely the medical balloon. The roll-sock sheath can be pulled back by withdrawing the outer layer of the roll-sock sheath, gradually to expose the medical balloon. A hydrophilic coating is provided on the outer surface of the outer layer of the roll-sock sleeve, which promotes the pushability of the balloon catheter assembly through a lesion. The coating makes it easier to push the distal end of the catheter assembly through a lesion, reduces friction between the lesion and the distal end of the balloon catheter assembly. The balloon remains protected by the roll-sock sleeve as does the coating of drug, which is exposed only just prior to deployment, once the balloon is in position.

### TECHNICAL FIELD

The present invention relates to a medical balloon assembly, a method of making a medical balloon assembly, a method of deploying a medical balloon, and to the use of a coated medical balloon assembly in treating vascular disease. The preferred embodiments relate to a coated medical balloon for use for example in angioplasty procedures, the balloon being coated with a bioactive agent advantageously selected to prevent restenosis of the vessel subjected to angioplasty.

### BACKGROUND OF THE INVENTION

Vascular atherosclerotic lesions are often localized at predetermined portions of blood vessels, where they cause constrictions or potentially occlusion of the vessel. Such lesions are typically treated by angioplasty using a catheter having an inflatable or expandable medical balloon at its distal end. The distal tip of the catheter is passed through the lesion so as to locate the balloon across the lesion, prior to expanding the balloon to open the vessel. Expansion will typically break any lesion material, such as sclerosis, fatty deposits and so on.

The balloon may be inflated and deflated a multitude of times to open the vessel and restore adequate blood flow. In some instances it may be necessary or advisable to fit a stent across the area of the legion in order to try to maintain long term arterial patency after angioplasty.

Another way in which blood vessels undergo stenosis is through disease. Probably the most common disease causing stenosis of blood vessels is atherosclerosis. Many medical devices and therapeutic methods are known for the treatment of atherosclerotic disease, including percutaneous transluminal angioplasty (PTA). During PTA, a balloon-tipped catheter is inserted in a patient's artery, the balloon being deflated. The tip of the catheter is advanced to the site of the atherosclerotic plaque to be dilated. The balloon is placed within or across the stenotic segment of the artery, and then inflated. Inflation of the balloon “cracks” the atherosclerotic plaque and expands the vessel, thereby relieving the stenosis, at least in part.

While PTA enjoys wide use, it suffers from two significant problems. First, the blood vessel may suffer acute occlusion immediately after or within the initial hours after the dilation procedure. Such occlusion is referred to as “abrupt closure” and may be caused by initial reactive hyperplasia. Abrupt closure occurs in perhaps five percent or so of the cases in which PTA is employed, and can result in myocardial infarction and death if blood flow is not restored promptly. The primary mechanisms of abrupt closure are believed to be elastic recoil, arterial dissection and/or thrombosis. It has been postulated that the delivery of an appropriate agent (such as an antithrombic) directly into the arterial wall at the time of angioplasty could reduce the incidence of thrombotic acute closure, but the results of attempts to do so have been mixed.

A second major problem encountered in PTA is the re-narrowing of an artery after an initially successful angioplasty. This re-narrowing is referred to as “restenosis” and typically occurs within the first six months after angioplasty. Restenosis is believed to arise through the proliferation and migration of cellular components from the arterial wall, as well as through geometric changes in the arterial wall referred to as “remodelling”. It has similarly been postulated that the delivery of appropriate agents directly into the arterial wall could interrupt the cellular and/or remodelling events leading to restenosis. However, like the attempts to prevent thrombotic acute closure, the results of attempts to prevent restenosis in this manner have been mixed.

Non-atherosclerotic vascular stenosis may also be treated by PTA. For example, Takayasu arthritis or neurofibromatosis may cause stenosis by fibrotic thickening of the arterial wall. Restenosis of these lesions occurs at a high rate following angioplasty, however, due to the fibrotic nature of the diseases. Medical therapies to treat or obviate them have historically been similarly disappointing.

Simple vessel opening by angioplasty, even with the addition of a stent is not always successful long term. In a significant proportion of patients, after some months patients can develop renewed narrowing of the vessel at the previous intervention point. Such narrowing, known as restenosis, is not necessarily caused by the formation of new atherosclerotic plaques but can be caused by cell hyperproliferation, particularly of the vascular smooth muscle cells, which is believed to be the result of the vessel dilatation and damage caused to the inner vessel wall (the tunica intima) by the foreign body, whether the balloon and/or stent. Restenosis may occur over a prolonged period of a few months but can also occur at the time of or very soon after angioplasty, by initial reactive hyperplasia.

It has been found that restenosis can be reduced or avoided by coating a medical balloon or a stent with an antiproliferative bioactive agent. A number of such agents have been found to be effective, including taxol (paclitaxel), limos based drugs such as rapamycin and so on.

The administration of such drugs has taken many forms over the development of the technology. Initially, it was understood that the antiproliferative drug must be released over a sufficiently long time span, so as to inhibit cell hyperproliferation over a period of months. Additionally, it was believed that drug loss into the patient's bloodstream had to be avoided, in particular in the case of a drug with high toxicity such as taxol. These considerations led to the development of drug eluting medical devices, including drug eluting balloons having, for example, a supply of drug that could be dispensed from within the structure of the balloon, for example from an internal conduit or outer layer of matrix material. Stents were developed with coatings that formed a matrix from which a drug could be discharged over time, or coatings over the drug layer that would release drug only after the coating has been ruptured, for instance. Other proposals housed the drug in an envelope, for instance microcapsules or micelles.

Later developments concluded that containment layers or other devices were unnecessary and risked creating other damage to the vessel wall tissue. It was also determined that it is important to apply drug to the vessel wall at the time of vessel dilation and not after a time delay, and that as a consequence containment or time release components intended to delay the time of application of the drug to the vessel wall were disadvantageous.

An optimum arrangement provides the drug as an outer layer of the medical device to permit direct application of the drug to the vessel wall with no material time delay and over a short period of time from one or more seconds to one or more minutes, with or without an excipient. Such application times are consistent with the time required to dilate a vessel, meaning that the drug can be administered simultaneously with vessel dilatation. Arrangements of this type can, however, suffer from significant loss of drug into the patient's bloodstream, with only a small proportion of drug in practice being applied to the vessel wall tissue.

The release of the drug can be dependent on the nature of the drug, in particular its hydrophilicity or hydrophobicity, the form in which the drug is provided, such as whether it is of crystalline or amorphous form, and whether or not it is provided with excipients or enhancers. Also of relevance to adhesion of the drug of the balloon surface and the speed of its administration is the nature of the balloon surface, for instance whether it has a smooth or slippery surface, a textured or roughened surface and so on.

Medical devices such as medical balloons, are typically deployed endoluminally from a remote percutaneous entry point, such as the femoral artery, through a catheter typically inserted as a first part of the clinical intervention. In some cases, the catheter is inserted into the patient's vasculature at the same time as the medical device assembly, typically over a guide wire. It is preferred for the introducer assembly to be as small as possible in terms of diameter, which facilitates its passage through the patient's vasculature. Problems can arise with maintaining the integrity of any bioactive material coating on the outer surfaces of a medical device, a balloon in this instance, particularly as a result of friction between the medical device and the overlying catheter. While these problems can be mitigated by holding the bioactive material within the device, including within a matrix layer, doing so suffers from the disadvantages mentioned above.

Practical difficulties can also be encountered when attempting to push the distal end of an introducer assembly through a lesion or closure in a vessel, for example through a stenosed portion of vessel.

Examples of introducer and medical device assemblies can be found, for instance, in U.S. Pat. Nos. 8,016,872, 8,603,036, 8,968,381, US-2019/0247210, US-2019/0298519, US-2020/0188645, US-2021/0186725, WO-2019/060930 EP-2,453,941 and WO-021076509.

### SUMMARY OF THE INVENTION

The present invention seeks to provide an improved medical balloon assembly, method of making a medical balloon assembly, method of deploying a medical balloon assembly and use of a coated medical balloon assembly in treating vascular disease. The preferred embodiments relate to a coated medical balloon for use for example in angioplasty procedures, coated with a bioactive agent advantageously selected to prevent restenosis of the vessel subjected to angioplasty.

According to an aspect of the present invention, there is provided a coated medical balloon assembly comprising:
a balloon catheter having a proximal end and a distal end, and a medical balloon mounted to the balloon catheter at or proximate the catheter distal end;
a bioactive material coating on at least the medical balloon;
a roll-sock sheath disposed over the medical balloon and at least a part of the balloon catheter, the roll-sock sheath comprising a first, inner, layer disposed adjacent the medical balloon and a second, outer, layer disposed over the first layer, the roll sock including first and second roll-sock sheath ends and a fold line, the first and second roll-sock sheath ends being disposed at a proximal location relative to the fold line, the fold line being disposed adjacent the distal end of the balloon catheter, the first roll-sock end being an end of the first roll-sock layer and the second roll-sock end being an end of the second roll-sock layer;
a coating of hydrophilic or lubricous material on an outer surface of the second roll-sock layer and forming an outer layer of the coated medical balloon assembly;
a roll-sock sheath retraction mechanism coupled to the second roll-sock end and configured to retract the second layer towards the proximal end of the catheter, whereby the first layer progressively wraps over itself outwardly to the second layer, thereby rolling back the roll-sock sheath and exposing the coated medical balloon for deployment.

The assembly provides a number of technical and clinical advantages. The roll-sock protects the bioactive material layer on the surface of the balloon. During deployment, the roll-sock can be everted, sliding its outer layer over the first, to uncover the balloon without there being any sliding of the roll-sock against the outer layer of the balloon. As a result, the risk of rubbing off bioactive material or otherwise disrupting the bioactive material layer is avoided. The roll-sock also protects the bioactive material layer during insertion of the balloon catheter into the patient, and in particular through the deployment catheter. The roll-sock can also protect the balloon and bioactive material layer thereon while the balloon is pushed into and through a vessel lesion, as the roll-sock maintains its protective configuration at this time. The roll-sock sleeve also adds strength and can add some rigidity to the distal end of the catheter balloon, giving it greater ability to push through a lesion. The outer hydrophilic or lubricous outer surface of the roll-sock also helps with the passage of the balloon catheter through the patient's vasculature and also through the lesion. As the hydrophilic or lubricous coating can be applied to the outer surface of the roll-sock, it will not be brought into contact with the balloon or the coating of the balloon, preventing any contamination of the bioactive material layer. Other advantages will become apparent to the skilled person from the teachings that follow.

There are many types of introducers. One type is what could be described as a push/pull introducer. Push/pull type introducers require pushing the medical device out of the distal end of the sheath or pulling the sheath in the proximal direction relative to the medical device. These introducers induce sliding interaction forces between the medical device and the sheath, which may contribute to a number of adverse effects. One is the sliding interaction forces between the drug coated medical device and the sheath, which may affect the integrity of the coating or may even rub off at least a part of the coating. Furthermore, the drug coating of the medical device may be sticky, resulting in a greater frictional force that must be overcome when using these types of introducers. Another reason is that the sliding interaction forces between the introducer and the medical device could adversely affect the integrity of the medical device, including being torn or strained. Furthermore, these frictional forces may cause the medical device, the balloon in this instance, to move during deployment, for example to be pulled backwards (in a proximal direction) or to jump forwards, in both cases risking the medical balloon not being deployed precisely at the necessary location in the vessel.

Splittable sheaths are known. However, while the have their uses, they are not considered optimal in the case of coated medical balloons, in light of risk of damaging the medical balloon during splitting of the sheath and of damaging or otherwise disturbing the bioactive coating on the medical device.

The assembly taught herein addresses these shortcomings with prior art systems.

It is envisaged that the roll-sock may be provided with a lubricious material to permit easy sliding of the outer layer of the roll-sock over the inner layer. In other embodiments, the roll-sock may be made of a low friction material, examples of which are provided below.

The outer layer of the roll-sock may have a larger diameter relative to the inner layer, to facilitate inversion of the inner liner. There may be provided a mechanism to split the roll-sock sleeve, preferably disposed at a proximal end of the introducer assembly and configured such that the roll-sock remains continuous, that is unsplit, at the location of the medical balloon. For this purpose, a weakened region may be formed in the tubular wall of the roll-sock. The weakened region can be oriented longitudinally and is preferably at least as long as the medical device, more preferably at least twice the length of the medical device, such that the length of roll-sock that is retracted to uncover the medical device can be split and collected in a space efficient manner, for example wound onto a retrieval spool or the like. The weakened region may in some embodiments be defined by a discontinuous structural layer extending longitudinally along the tubular wall of the outer layer of the roll-sock sleeve.

Advantageously, the assembly comprises a dilator tip at the distal end of the balloon catheter, the roll-sock sheath fold line being disposed adjacent the dilator tip when the roll-sock sheath is in a balloon overlying configuration, the coating of hydrophilic or lubricous material being disposed over the second roll-sock layer and the dilator tip.

Preferably, the first layer of the roll-sock sheath contacts the medical balloon when the roll-sock sheath is in a balloon overlying configuration. Such contact can ensure the integrity of the balloon and bioactive material coating are maintained during deployment as well as during storage.

Preferably, the first end of the roll sock sheath is disposed at or adjacent a proximal end of the coated balloon.

The first end of the roll sock sheath is advantageously attached to the catheter at or adjacent the proximal end of the coated balloon.

The second end of the roll sock sheath may be disposed at one of: (a) a location at or adjacent a proximal end of the catheter; and (b) an intermediate location of the catheter between its proximal and distal ends.

The assembly preferably comprises a roll-sock sheath winding mechanism operable to wind the second end of the roll-sock sheath during retraction thereof. It may also include a roll-sock splitting device configured to split the second end of the roll-sock sheath longitudinally prior to winding on the winding mechanism, wherein the splitting mechanism splits the roll-sock sheath only at or adjacent the proximal end of the balloon catheter.

The system may include a handle disposed at the proximal end of the balloon catheter, configured to pull back the roll sock sleeve. For the purposes described above, the handle may include a splitter configured to slice the wall of the outer catheter longitudinally in a distal direction to form a sliced portion of the outer sleeve. The handle may also include a rotatable mechanism attached to the sliced portion of the outer sleeve. Rotation of the rotatable mechanism retracts a portion of the outer sleeve and winds the sliced portion of the outer layer of the sleeve about the rotatable mechanism, which may be a spindle.

Preferably, there is provided a low friction material disposed between the first and second layers of the roll-sock sheath. The low friction material may comprise one or more of: a material forming the roll-sock sheath, a layer of low friction compound between the first and second layers of the roll-sock sheath, a liquid or gas disposed between the first and second layers of the roll-sock sheath.

The layer of low friction compound may be a layer of hydrophilic material.

In some embodiments, a liquid or gas may be disposed between the first and second layers at the time of retraction of the roll-sock sheath, the apparatus comprising a device for locating such liquid or gas between the first and second layers of roll-sock sheath at the time of retraction.

According to another aspect of the present invention, there is provided a method of deploying a coated medical balloon in a patient comprising:
providing a balloon catheter having a proximal end and a distal end, and a medical balloon mounted to the balloon catheter at or proximate the catheter distal end; a bioactive material coating on at least the medical balloon; a roll-sock sheath being disposed over the medical balloon and at least a part of the balloon catheter, the roll-sock sheath comprising a first, inner, roll-sock layer disposed adjacent the medical balloon and a second, outer, roll-sock layer disposed over the first roll-sock layer, the roll-sock sheath including first and second roll-sock sheath ends and a fold line, the first and second roll-sock sheath ends being disposed at a proximal location relative to the fold line, the fold line being disposed adjacent the distal end of the balloon catheter, the first roll-sock sheath end being an end of the first roll-sock layer and the second roll-sock sheath end being an end of the second roll-sock layer; a coating of hydrophilic or lubricous material disposed over the second roll-sock layer and forming an outer layer of the coated medical balloon assembly;
feeding the assembly endoluminally through the vasculature of a patient, whereby the hydrophilic or lubricous coating eases the passage of the assembly through the vasculature;
retracting the second roll-sock layer, whereby the first roll-sock layer progressively wraps over itself outwardly to the second roll-sock layer to roll back the roll-sock sheath and expose the medical balloon for deployment, and whereby the hydrophilic or lubricous layer overlying the second roll-sock layer is retracted with the second roll-sock layer and such that contact between the hydrophilic or lubricous layer on the roll-sock sheath and the bioactive material layer on the balloon is avoided or minimised.

Advantageously, a dilator tip disposed at the distal end of the balloon is coated with the hydrophilic or lubricous material disposed over the second roll-sock layer, and wherein the step of feeding the assembly endoluminally through the vasculature of a patient is eased by the hydrophilic or lubricous coating over the roll-sock sheath and the dilator tip.

Preferably, the first roll-sock layer of the roll-sock sheath contacts the medical balloon when the roll-sock sheath is in a balloon overlying configuration.

The method may comprise winding the second end of the roll-sock sheath during retraction thereof. It may comprise splitting the second end of the roll-sock sheath longitudinally prior to winding on the winding mechanism, wherein the splitting mechanism splits the roll-sock sheath only at or adjacent the proximal end of the balloon catheter.

Advantageously, the method comprises disposing a low friction material between the first and second roll-sock layers of the roll-sock sheath. The low friction material may comprise one or more of: a material forming the roll-sock sheath, a layer of low friction compound between the first and second roll-sock layers of the roll-sock sheath, a liquid or gas disposed between the first and second layers of the roll-sock sheath. The method may comprise the step of disposing the liquid or gas between the first and second layers at the time of retraction of the roll-sock sheath.

According to another aspect of the present invention, there is provided the use of a coated medical balloon assembly comprising:
a balloon catheter having a proximal end and a distal end, and a medical balloon mounted to the balloon catheter at or proximate the catheter distal end;
a bioactive material coating on at least the medical balloon; a roll-sock sheath disposed over the medical balloon and at least a part of the balloon catheter, the roll-sock sheath comprising a first, inner, roll-sock layer disposed adjacent the medical balloon and a second, outer, roll-sock layer disposed over the first layer, the roll-sock sheath including first and second roll-sock sheath ends and a fold line, the first and second roll-sock sheath ends being disposed at a proximal location relative to the fold line, the fold line being disposed adjacent the distal end of the balloon catheter, the first roll-sock end being an end of the first roll-sock layer and the second roll-sock end being an end of the second roll-sock layer; a coating of hydrophilic or lubricous material disposed over the second roll-sock layer and forming an outer layer of the coated medical balloon assembly; a roll-sock sheath retraction mechanism coupled to the second roll-sock sheath end and configured to retract the second roll-sock layer, whereby the first roll-sock layer progressively wraps over itself outwardly to the second roll-sock layer, thereby rolling back the roll-sock sheath and exposing the medical balloon for deployment;
in the deployment of a medical balloon in the vasculature of a patient to deliver one or more bioactive materials to vessel tissue for the treatment of a medical condition.

According to another aspect of the present invention, there is provided a method of making a coated medical balloon assembly comprising the steps of:
applying at least one layer of bioactive material coating on at least a medical balloon of a balloon catheter having a proximal end and a distal end, and a medical balloon mounted to the balloon catheter at or proximate the catheter distal end;
disposing a roll-sock sheath over the medical balloon and at least a part of the balloon catheter, the roll-sock sheath comprising a first, inner, roll-sock layer disposed adjacent the medical balloon and a second, outer, roll-sock layer disposed over the first roll-sock layer, the roll-sock sheath including first and second roll-sock sheath ends and a fold line, the first and second roll-sock sheath ends being disposed at a proximal location relative to the fold line, the fold line being disposed adjacent the distal end of the balloon catheter, the first roll-sock end being an end of the first roll-sock layer and the second roll-sock end being an end of the second roll-sock layer;
applying a coating of hydrophilic or lubricous material over the second roll-sock layer, the hydrophilic or lubricous material coating forming an outer layer of the coated medical balloon assembly;
providing a roll-sock sheath retraction mechanism coupled to the second roll-sock sheath end and configured to retract the second roll-sock layer towards the proximal end of the catheter, whereby the first roll-sock layer progressively wraps over itself outwardly to the second roll-sock layer, thereby rolling back the roll-sock sheath and exposing the coated medical balloon for deployment.

Preferably, the medical balloon is used for opening a vessel. The use is preferably in the treatment of one of: stenosis, restenosis, reactive hyperplasia, thrombosis, cancerous tissue.

Advantageously, the medical balloon is coated in an inflated or expanded condition.

The assembly is advantageously free of any stent on the balloon, that is there is no stent disposed on the balloon.

In all aspects, the layer of bioactive material may:
a) consist of or be principally of bioactive material;
b) be or include a therapeutic substance;
c) be or include an anti-proliferative bioactive substance; or
d) be or include paclitaxel or other bioactive agent.

In the preferred embodiments of all aspects, the bioactive material layer is free of one or more of:
a) containment elements;
b) binding agents; and
c) time control release agents;
d) polymer or other matrix material.

The preferred embodiments relate to a balloon catheter having no overlying medical device, in particular a stent.

It is to be understood that the lubricious outer coating on the roll-sock sleeve may be provided by a separate layer applied to (coated onto) the assembly, as shown in the preferred embodiments. It is to be understood, though, that this lubricious outer coating may be an integral part of the roll-sock sleeve, for example constituted by the material forming the roll-sock sleeve, or by an outer layer thereof.

Other aspects and advantages of the teachings herein will become apparent to the person skilled in the art from the disclosure of the preferred embodiments that follows.

### BRIEF DESCRIPTION OF THE DRAWINGS

Embodiments of the present invention are described below, by way of example only, with reference to the accompanying drawings, in which:

FIG. 1 is a schematic view of an exemplary medical balloon that can be coated with a bioactive material layer and can be deployed in accordance with the teachings herein;

FIG. 2 is a schematic diagram of a preferred embodiment of introducer assembly according to the present invention;

FIG. 3 is a schematic diagram of the distal end of the introducer assembly of FIG. 2 disposed in a vessel;

FIG. 4 is an enlarged view of a part of the distal end of the introducer assembly of FIG. 3;

FIG. 5 is a schematic diagram of the distal end of the introducer assembly of FIGS. 2 to 4 in the course of deploying the medical balloon;

FIG. 6 is an enlarged view of a part of the distal end of the introducer assembly similar to that of FIG. 4, in the course of deploying the medical balloon;

FIG. 7 is a schematic diagram of the distal end of the introducer assembly of FIGS. 2 to 6 with the medical balloon in an inflated or expanded condition in a vessel; and

FIG. 8 is an enlarged view of a part of the distal end of the introducer assembly similar to that of FIGS. 4 and 6 with the medical balloon in an inflated or expanded condition in a vessel.

### DESCRIPTION OF THE PREFERRED EMBODIMENTS

For the purposes of understanding the principles taught herein, reference will now be made to the embodiments illustrated in the drawings, and specific language will be used to describe the same. It should nevertheless be understood that no limitation of the scope of the claims is thereby intended, such alterations and further modifications in the illustrated device, and such further applications of the principles taught herein being contemplated as would normally occur to one skilled in the art to which the present invention relates.

It is to be understood that the drawings are schematic only and not to scale. Often only the principal components relevant to the teachings herein are shown in the drawings, for the sake of clarity.

In the following, the terms “proximal” and “distal” are used to describe the opposing axial ends of the introducer system. The term “proximal” is used in its conventional sense to refer to the end of the apparatus (or component thereof) closest to the operator during use of the apparatus. The term “distal” is used in its conventional sense to refer to the end of the apparatus (or component thereof) that is initially inserted into the patient, or that is closest to the patient during use.

The terms bioactive agent and bioactive material are used interchangeably in this specification and are intended to encompass materials, elements and compounds that have a therapeutic and/or preventative effect, typically drugs. They may, for example, be or include a therapeutic substance, such as an anti-proliferative bioactive substance of which paclitaxel is a preferred agent. Further examples of bioactive agents are given below and will also be apparent to the person skilled in the art.

The term “an immediate release and bioavailability” is meant to refer to a release from the balloon surface in periods of time ranging between 1 second and 1.5 minutes, preferably between 20 seconds and 1 minute, and an absorption by the vascular tissue in periods of time ranging between 1 second and 25 minutes, preferably between 20 seconds and 25 minutes.

The term “therapeutically effective amount” means a drug amount capable of inducing a therapeutical or preventive effect against the restenosis of the treated vascular tissue in the patient.

The terms “expanded” and “inflated” and equally “expandable” and “inflatable” in relation to the medical balloon are intended to be interchangeable.

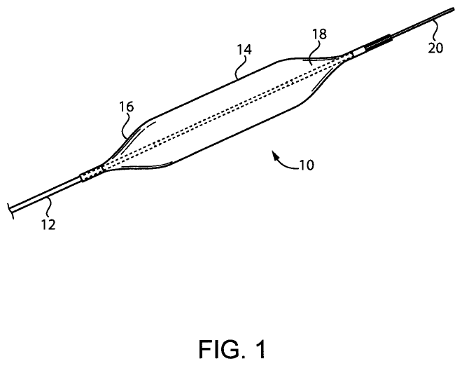

Referring first to FIG. 1, this shows an exemplary medical balloon 10 that may be used for angioplasty procedures, for deployment of a medical device such as a stent or stent graft, for valvuloplasty procedures or the like. The medical balloon is fitted to a balloon catheter 12 and has a substantially cylindrical balloon body 14 terminating in end cones 16, 18 that taper towards the balloon catheter 12 and fix the balloon wall to the catheter in fluid-tight manner. The balloon catheter 12 may include a lumen therein for the passage of a guide wire 20, as well as a lumen (not shown) for providing inflation fluid into the balloon. The basic structure of the balloon 10 may be of a type conventional in the art, prior to modification by the teachings herein. Although FIG. 1 depicts a simple balloon structure, it may have any of the features known for such balloons, including a different shape, surface roughening, texturing, a slip coating, and so on.

The balloon 10 may be folded for deployment, around the catheter 12, and for this purpose may be formed with pre-folds, which assist the folding of the balloon during manufacture and assembly and also in the collapse of the balloon after the treatment to assist in the removal of the balloon catheter 12 from the patient post-procedure.

An angioplasty balloon of the type depicted schematically in FIG. 1 is often able to open a closed vessel in a very short period of time, for instance in seconds or minutes. While the initial vessel opening procedure is fast, there is a significant risk of future closure of the vessel, for instance by repeated collapse or restenosis. This can be caused by a number of factors including reactive hyperplasia resulting from the vessel opening procedure. Vessel closure can occur again within a few weeks or months of the medical procedure.

In the examples described briefly above in connection with FIG. 1, it has been found that the administration of suitable bioactive agents into the vessel wall from the stent and/or from the medical balloon can substantially retard or prevent subsequent closure of the vessel due to restenosis. A variety of bioactive agents suitable for such purposes are known in the art including, for instance, antithrombogenic agents, thrombin inhibitors, tissue plasminogen activators, thrombolytic agents, fibrinolytic agents, vasospasm inhibitors, antiplatelet agents, antiproliferative agents and so on. A particularly effective bioactive agent known in the art is paclitaxel, other examples include dexamethasone, rapamycin, analogues of rapamycin such as Sirolimus, heparin and numerous other agents and compounds and analogues thereof.

Specifically, the bioactive material of the coating may include at least one of: paclitaxel and/or paclitaxel derivatives, rapamycin and/or rapamycin derivatives, docetaxel and/or docetaxel derivatives, cabazitaxel and/or cabazitaxel derivatives, taxane and/or taxane derivatives, estrogen or estrogen derivatives; heparin or another thrombin inhibitor, hirudin, hirulog, argatroban, D-phenylalanyl-L-poly-L-arginyl chloromethyl ketone or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; an antimicrobial agent or antibiotic; aspirin, ticlopidine or another antiplatelet agent; colchicine or another antimitotic, or another microtubule inhibitor; cytochalasin or another actin inhibitor; a remodelling inhibitor; deoxyribonucleic acid, an antisense nucleotide or another agent for molecular genetic intervention; GPIIb/IIIa, GP Ib-IX or another inhibitor or surface glycoprotein receptor; methotrexate or another antimetabolite or antiproliferative agent; an anti-cancer chemotherapeutic agent; dexamethasone, dexamethasone sodium phosphate, dexamethasone acetate or another dexamethasone derivative, or another anti-inflammatory steroid; dopamine, bromocriptine mesylate, pergolide mesylate or another dopamine agonist; 60Co (having a half-life of 5.3 years), 192Ir (73.8 days), 32P (14.3 days), 111In (68 hours), 10 90Y (64 hours), 99mTc (6 hours) or another radio therapeutic agent; iodine containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting 15 enzyme (ACE) inhibitor; ascorbic acid, alphatocopherol, superoxide dismutase, deferoxamine, a 21-amino steroid (lasaroid) or another free radical scavenger, iron chelator or antioxidant; angiopeptin; a 14C-, 3H-, 131|1-, 32P- or 36S-radiolabelled form or other radio labelled form of any of the foregoing; or a mixture of any of these.

The bioactive material is coated onto the balloon 10 so as to be released from the balloon 10 into the tissues of the vessel, and should be dispensed at a rate suitable for treating the required medical condition. In the case of a medical device which is temporarily deployed in a patient's vessel, such as an angioplasty balloon, the bioactive agent optimally released from the balloon in a very short period of time, for instance within seconds or minutes, although sometimes up to an hour or more.

It is advantageous that the bioactive agent is held onto the medical device during deployment of the device in the patient without excessive loss of bioactive material into the patient's bloodstream, for instance. For this purpose, the prior art has suggested restraining the bioactive material, for instance in a containment or time release layer or matrix. Examples include: porous polymer layers into which bioactive material can be embedded, enclosed chambers holding the bioactive material, outer coatings disposed over the bioactive material and which dissolve or open during the deployment process, encapsulation of the bioactive material in micelles, capsules or pellets, and so on. Such containment measures can, however, lead to a number of disadvantages, including undesirable delayed administration of the bioactive material into body tissues, presence of a foreign substance in the body, possible onset of stenosis caused by the carrier mechanism itself, and so on.

The inventors have found that the optimal solution is to apply the bioactive agent in the absence of any containment or time release substance and from a layer that is predominantly or entirely made of bioactive agents. In this manner, after administration of the bioactive agent or agents, the medical device remains free of agent delivery substances (polymer layers, for example) and no unnecessary carrier substances are released into the patient's body. A difficulty, however, has existed with getting the bioactive agent(s) to be held sufficiently well on the medical device.

In the case of medical balloons and other short term use medical devices, it is generally preferred that the bioactive agent is released quickly into the patient's tissue. For this purpose, an excipient such as urea and/or urea derivatives, gallates and gallate derivatives (such as epigallocatechin gallate), saccharides and/or saccharide derivatives, chitin and/or chitin derivatives, ascorbic acid, citric acid, stearates and/or stearate derivatives, polyvinyl pyrrolidone, dicalcium phosphate dihydrate, eudragit polymers and/or eudragit polymers derivatives, cellulose and/or cellulose derivatives, PEG, polysorbate 80, sodium lauryl sulphate, chitosan, magnesium dioxide, silicon dioxide, carbonate derivatives, plasdone, butylated hydroxyanisole, succinic acid, sodium dioctyl sulfosuccinate, precirol ATO 5, may be added to the bioactive agent layer or disposed so as to underlie the bioactive material layer. The excipient will speed up the release of the bioactive agent once the medical device is deployed within the patient, for instance by the excipient dissolving within the patient's blood plasma and providing for quick release of the bioactive agent. This can be particularly useful in treating initial reactive hyperplasia occurring as a result of angioplasty or other medical procedures. When an excipient is used, this may be as a sublayer between the layer of bioactive material and the medical device or as a layer above the layer of bioactive material. The excipient acts to speed the release of the bioactive agent (drug for example), compared to a drug per se or a drug held in a containment or time release layer. In the case of a sublayer of excipient, the functionalisation of the surface to be coated will be matched to the nature of the excipient and the excipient matched to the bioactive agent or agents.

The bioactive agent may be in amorphous or crystalline form or any mixture thereof.

There may be provided a single layer of bioactive material or multiple layers.

The bioactive material can be any of a large variety and many bioactive materials for coating medical devices are known in the art. The layer of bioactive material applied to the surfaces of the device may be of a single bioactive material or a combination of different bioactive agents, in dependence upon the desired treatment.

The balloon is preferably inflated before coating with the bioactive agent and then it is folded. The balloon can be completely or partially coated with bioactive agent. Coating may be by dipping, spraying, or depositing by means of a syringe, a micropipette, or other similar dispensing device; or by any other suitable method.

The bioactive material layer is preferably one of:
a) wholly or principally of bioactive material;
b) wholly or partially a therapeutic substance;
c) wholly or partially an anti-proliferative bioactive substance; and
d) wholly or partially paclitaxel, dexamethasone, rapamycin or Sirolimus.

The bioactive material layer is preferably free of one or more of:
a) containment elements;
b) binding agents;
c) time control release agents; and
d) polymer or other matrix material.

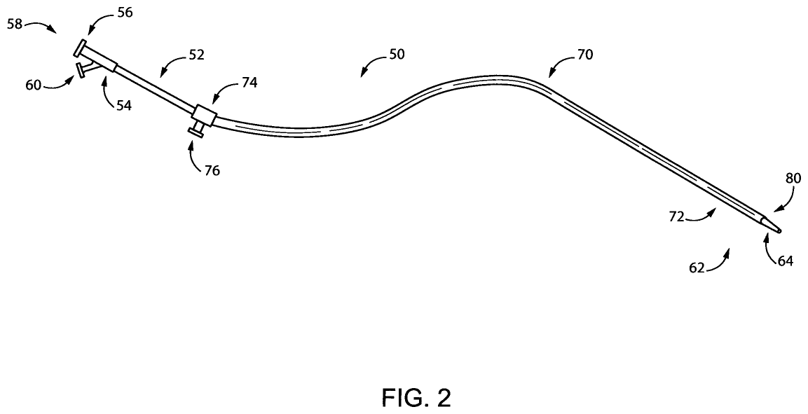

Referring now to FIG. 2, this shows a schematic drawing of a preferred embodiment of introducer assembly 50 according to the present invention. Only the principal components of the assembly 50 are shown in FIG. 2 and described herein, as the other components of such assemblies will be familiar to the skilled person and are not necessary for an understanding of the invention taught herein.

The assembly 50 includes a balloon catheter shaft 52 having a proximal end 54 at which there is provided a Y-shaped connector 56 with first and second Luer connector ports 58,60. The balloon catheter shaft 52 has a distal end 62, at which there is fitted a dilator tip 64. A medical balloon (not visible in FIG. 2) is located at or adjacent the distal end 62 of the catheter shaft 50, preferably at the zone 66 shown in FIG. 2. The catheter shaft 52 typically includes a guide wire lumen extending from and through the Luer connector 58 and through a lumen in the dilator tip 64, enabling the assembly 50 to be deployed over a guide wire, the latter typically being introduced endoluminally through the patient's vasculature from a remote percutaneous entry point, such as the femoral artery. The guide wire can be inserted by any suitable technique, the Seldinger being common.

The guide wire catheter 52 also includes a second lumen coupled to the side branch port 60 of the Y-fitting 56 and extends to a portion of the catheter shaft 52 located within the zone of the medical balloon, at which location there is an exit aperture from that lumen to the internal balloon chamber. This lumen, which could usefully be characterised as an inflation/deflation lumen, used for the passage of inflation fluid to inflate or expand the balloon, and also for with drawing inflation fluid out of the balloon to allow the balloon to collapse. For this purpose, the balloon, as previously described, may be made of an expandable material, that is a stretchable material, or may be made of a flexible and foldable material such as that upon inflation the balloon unfolds and unfurls to its expanded condition. Upon withdrawal of inflation fluid, the balloon can collapse from its inflated state and be furled and/or folded again. For this purpose, the balloon could have pre-formed fold lines, generated during initial manufacture of the balloon, to assist in the collapse and folding of the balloon.

Disposed on the outside of the balloon catheter shaft 52, and the medical balloon (not shown in FIG. 2), is a sheath 70 having a roll-sock portion 72, described in further detail below, including at its proximal end 74 a side port 76. The sheath 70 is in at least some embodiments formed of two layers able to slide over one other in a roll-sock manner, the two layers extending to the proximal end 74 of the sheath 70 and in practice of the assembly 50. The side port 76 can be used to deliver into the space between the inner and outer layers of the roll-sock sheath 70 a lubricating material, such as a liquid or gas, for reducing any friction between the two layers of the roll-sock sheath 70, as all will be described in further detail below.

The roll-sock sheath 70 extends to a distal point 80 which, before employment of the medical balloon, preferably extends to and most preferably over at least a portion of the dilator tip 64 and covering completely the medical balloon. At the distal point 80 there is a practice of a fold line between the inner and outer layers of the roll-sock sheath 70. As will be described in further detail below, the roll-sock sheath 70 can be pulled back by withdrawing, in a proximal direction, the outer layer of the roll-sock sheath 70, gradually to expose the medical balloon located in the zone 66 of the assembly 50.

The catheter shaft 52 may be constructed from one or more of materials commonly used in the art, including but not limited to: polyurethane, PTFE (including ePTFE and siliconized PTFE), high density polyethylene (HDPE), polyamide, polyimide, and so on.

The roll-sock sheath 70 may be made of a uniform layer and structure of material and may be made of a low friction of lubricous material which presents a slippery luminal surface to allow easy rolling of the sheath 70 over itself. The wall of the roll-sock sheath 70 can have sufficient strength to protect the medical balloon during deployment within the catheter.

PTFE is a preferred material for the roll-sock sheath 70. PTFE can have suitably high longitudinal tensile strength which permits the sheath 70 to be longitudinally rolled with higher forces, and a sufficient transverse tensile strength to retain the tubular medical device in the compressed configuration. PTFE also provides structural integrity and durability at a lower thickness up to 0.05 millimetres.

Other polymeric materials or resins usable for the roll-sock sheath 70, and the catheter shaft 52 if desired, include hydrophilic polyurethanes, aromatic polyurethanes, polycarbonate base aliphatic polyurethanes, engineering polyurethane, elastomeric polyamides, block polyamide/ethers, polyether block amide (PEBA), including PEBAX®, silicones, polyether-esters, polyether-ester elastomers, including Arnitel® (DSM Engineering Plastics), nylons, polyesters, polyester elastomers, including Hytrel® (Du Pont), linear low density polyethylenes, such as Rexell®, and combinations thereof.

The balloon material preferably includes a polyamide such as nylon or nylon 12 for applying the bioactive material directly thereto. A hydrophilic slip coating can be applied to the surface of the medical balloon to further facilitate the delivery and attachment of the bioactive material to the vessel wall. Other balloon materials include PEBAX, polyethylene or irradiated polyethylene which has a smooth or slippery surface.

Where the medical balloon would benefit from fast release of the bioactive material the coating may include or overlie an excipient.

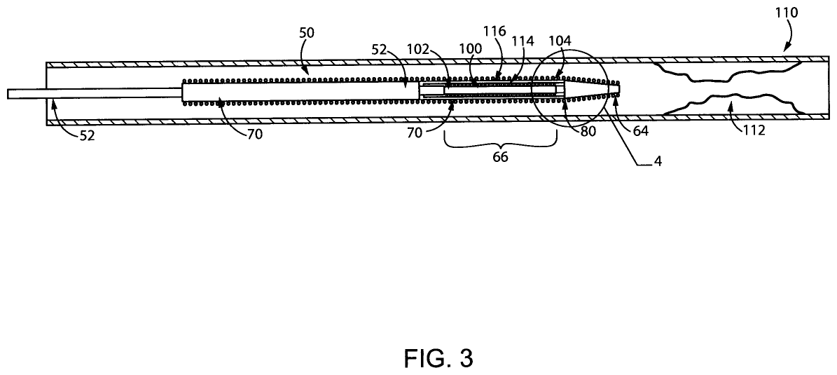

Referring now to FIG. 3, this shows in schematic form a portion of the distal end of an introducer assembly 50 similar to that of FIG. 2, disposed within a vessel 110. Typically, the vessel 110 will include a lesion 112 obstructing the vessel 110 and obstructing or restricting normal flow of blood (or other fluid) through the vessel 110. The treatment, as described as above, is to locate an angioplasty balloon, such as the balloon 100 shown in FIG. 3, across the lesion 112. The balloon is then inflated, one or more times, to remove the lesion and open the vessel. For this purpose, the balloon 100 may be provided with cutting or scoring elements of a type known in the art and as disclosed, for example, US-9,339,291, U.S. Pat. No. 5,209,799, GB-2,485,769, GB-2,487,400, GB-2,520,727. A cutting or scoring balloon can be used to break away plaque or other stenosis material forming the lesion in order to open the vessel 110.

It is to be appreciated that FIG. 3 shows the medical balloon assembly in schematic form only. For instance, the roll-sock sleeve 70 will in practice extend to the proximal end of the catheter assembly 50 and will not terminate half way along the catheter assembly 50. The outer layer of the roll-sock sleeve 70 or at least a part or component of it, will extend to the proximal end of the assembly 50 so that it can be pulled by the clinician to cause the sleeve 70 to roll, in effect backwards (that is proximately), to uncover the medical balloon for deployment thereof.

FIG. 3 also shows the distal end of the roll-sock sleeve, extending over the region 66 in a part-cutaway view such that the medical balloon 100 is visible. In this example, the roll-sock sleeve 70 includes a first or inner layer 114, adjacent medical balloon 100, and a second or outer layer 116 that overlies the inner layer 114 and is formed by eversion of the sleeve 70 over itself at the fold line 80, which in this example is disposed adjacent to the proximal end of the dilator tip 64.

The medical balloon 100 includes a proximal end 102 and a distal end 104, both of which are fixed in sealed manner to the catheter shaft 52, in a similar manner to the example balloon shown in FIG. 1. The distal end 104 of the balloon 100 is preferably spaced from the proximal end of the dilator tip by a short distance. Internally within the space occupied by the balloon 100, there is a port or opening coupled to the balloon inflation lumen as disclosed above.

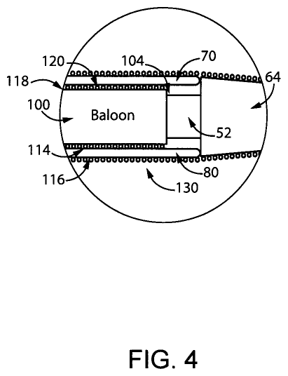

An enlarged view of a part of the assembly of FIG. 3 is shown in FIG. 4, specifically the distal end of the balloon 100 and the proximal end of the dilator tip 64. In this image, the inner and outer layers 114,116 of the roll-sock sleeve 70 can be clearly seen. In practice, the inner layer of 114 of the roll-sock sleeve 70 has a proximal end which need not terminate at the proximal end of the assembly 50 but could terminate at a location proximal but close to the proximal end of the medical balloon 100, given that the inner layer 114 of the roll-sock sleeve 70 does not need to slide at all relative to the balloon and does not need to be everted beyond the proximal end of the balloon, as once the balloon is released from the sleeve 70, by rolling back of the fold line 80 beyond the proximal end of the balloon 100, the inner layer of the sleeve 70 has no further effective purpose. It is not excluded, however, that in some embodiments the proximal end of the inner layer 114 of the roll-sock sleeve 70 could extend all the way to the proximal end of the assembly 50, in particular to the side port assembly 76, if desired. However, in most cases, a fluid tight coupling can be achieved between the catheter sheath 52 and the outer layer 116 of the roll sock sleeve, and also by the inner layer 114 and its fixation to the catheter sleeve, thereby providing an enclosed space for feeding and holding a lubricating material between the two layers 114,116 of the roll-sock sleeve 70.

Depicted also in FIG. 4 is a layer 120 (shown schematically as a line of dots) of bioactive material coating the outer surface of the balloon 100. Advantageously, and as described above, the bioactive material layer 120 is applied to the balloon during manufacture with the balloon in an expanded of inflated condition, so as to extend across the whole circumference of the balloon, and also its length, as a uniform layer of bioactive material 120. It is to be understood that the coating 120 of bioactive material on the surface of the balloon could take a variety of forms, as described above and a particular could be a single of bioactive layer material or multiple layers of bioactive material, and may also comprise if desired an excipient to assist in the release of bioactive material from the balloon. Such excipient could be incorporated in the layer of bioactive material or could be an underlying layer, for example. The bioactive material may comprise a single bioactive agent, as described above, or a plurality of bioactive agents in accordance with the clinical need.

Additionally, the bioactive material could be of any suitable format, such as in amorphous form, crystalline form or any mixture. Reference is made to the above description of suitable bioactive agents.

FIG. 4 also shows the provision of a hydrophilic or lubricous coating 130 (schematically shown as a line of dots) on the outer surface of the roll-sock sleeve 70 specifically the outer surface of the outer or second layer 116 of the roll-sock sleeve 70, and preferably also extends along the outer surface of the dilator tip 64. The hydrophilic or lubricous coating 130 promotes the pushability of the balloon catheter assembly through the patient's vasculature and particularly also through a lesion 112. The hydrophilic or lubricous coating layer has a number of useful advantages. Not only does it make it easier to push the distal end of the catheter assembly 50 through a lesion 112 without requiring less pushing force, it also reduces friction between the lesion 112 and the distal end of the balloon catheter assembly 50 so as to reduce any possible friction damage or other friction effects on the catheter assembly 50 as it is pushed through the lesion 112. The hydrophilic or lubricous coating 130 can also assist in the pushability and trackability of the catheter assembly 50 through the patient's vasculature as it is deployed to the treatment side. As will be apparent from FIG. 4 in particular, the hydrophilic or lubricous coating 130 does not extend to the inner roll-sock sleeve layer 114, so there is no hydrophilic or lubricous material that comes into contact with the bioactive material coating layer 120 on the balloon 100. During operation, as the outer layer 116 is pulled backwards, that is in a proximal direction, the inner layer 114 will be progressively everted (at junction 80), which will occur as the hydrophilic or lubricous coating layer 130 on the roll-sock sleeve 70 is pulled backwards, in practice moving the hydrophilic or lubricous coating 130 away from the balloon with a progressively increasing length of inner sleeve layer 114 being everted to the outer layer of the roll-sock sleeve 70. This can help to ensure that none of the hydrophilic or lubricous coating 130 on the roll-sock sleeve 70 comes into contact with the bioactive material of the layer 120.

In other embodiments, the roll-sock sleeve 70 itself may be made of a lubricous, or low friction material so that the material of the roll-sock sleeve 70 itself provides that low friction coating. In some embodiments, the part of the roll-sock sleeve 70 which forms the outer layer 116 in its most extended position (that is the configuration shown in FIGS. 1 to 3) may be made of a low friction material, while the inner layer 114 may be formed of a different material, if so desired.

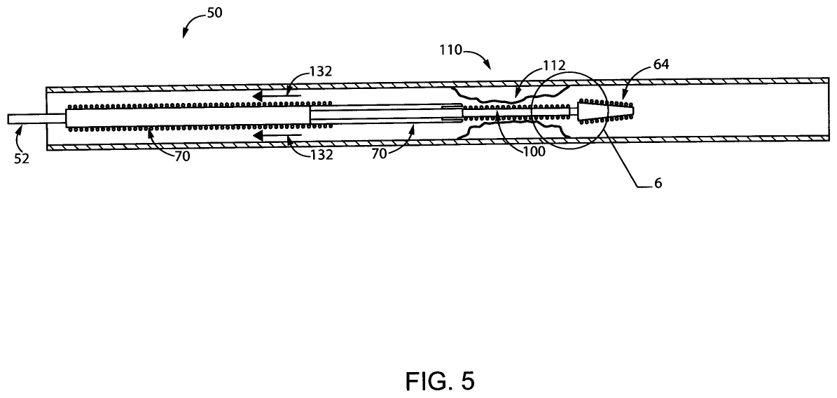

Referring now to FIG. 5, this shows a view of a part of the introducer assembly, similar to that of FIG. 3, and in which the distal end of the introducer assembly 50 has been passed through the lesion 112 in the vessel 110. Additionally, the roll-sock sleeve 70 has been retracted, by pulling the outer sleeve layer 116 in a proximal direction towards the proximal end of the assembly 50, such that the balloon 100 is no longer covered by the roll-sock sleeve 70 and is exposed for deployment.

It will be appreciated, considering FIGS. 3 and 5 together, that the introducer assembly will be pushed through the lesion 112 with the roll-sock sleeve 70 in its original, that is most extended, condition, such that the medical balloon 100 remains within and protected by the roll-sock sleeve 70 during the passage of the distal end of the assembly 50 through the lesion 112. As a result, the balloon 100 remains protected by the roll-sock sleeve 70 as it is pushed through the lesion 112. The hydrophilic or lubricous exterior layer, a coating in this embodiment, on the exterior of the roll-sock sleeve 70, and preferably also on the dilator tip 64, eases the pushing of the balloon 100 through the lesion 112 with reduced friction.

Once it has been determined that the balloon 100 is correctly positioned across the lesion 112, achieved by any known method such as fluoroscopy, the roll-sock sleeve 70 can be retracted to the configuration shown in FIG. 5. During this process it is preferred that the balloon 100 is maintained in its unexpanded, that is uninflated, configuration.

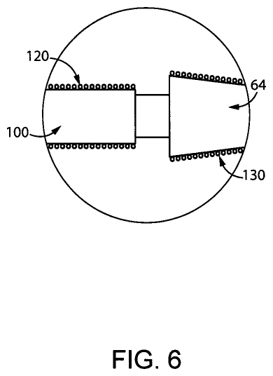

With reference to FIG. 6, this shows a portion of the distal end of the assembly 50 identified by the circle in FIG. 5. This Figure shows a part of the dilator tip 64, preferably still comprising the lubricating or low friction coating 130, and a portion of the balloon 100 with the bioactive material layer 120 over the balloon. As a result of the protection provided by the roll-sock sleeve 70, the integrity of the bioactive material layer 120 is maintained. It will be appreciated also that as the roll-sock sleeve 70 is withdrawn by pulling back on the outer layer 116 without disturbing the position of the inner layer 114 relative to the balloon 100, save for its eversion as the outer layer 116 is pulled backwards. There is no scraping of the roll-sock sleeve against the balloon. Additionally, as the roll-sock sleeve is retracted in an intact manner, that is, it is not split or otherwise damaged, the roll-sock sleeve 70 will always present a uniformly circular contact surface against the balloon 100, albeit a layer which progressively reduces in length and coverage over the balloon as the roll-sock sleeve is everted and pulled away from the balloon. This again ensures maintenance of the integrity of the bioactive material layer 120 on the outer surface of the balloon 100.

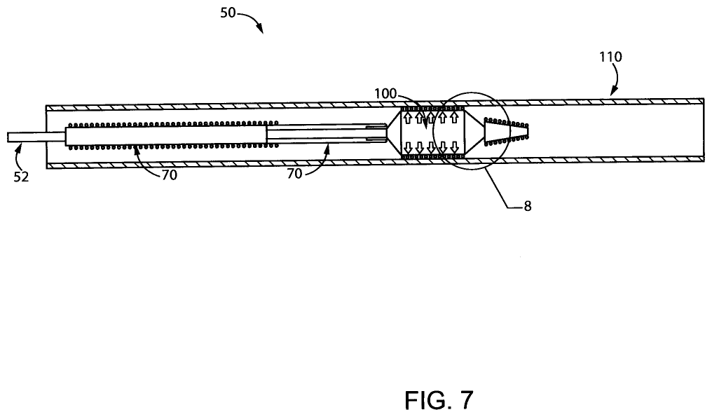

Referring now to FIG. 7, this shows a view similar to that of FIG. 5, namely of the distal end of the assembly 50, in which the medical balloon 100 has been inflated or expanded, by feeding into the chamber of the balloon 100 inflation fluid through the inflation lumen and port 60 of the assembly 50. Inflation of the balloon 100 compresses the lesion 112 to the vessel wall, effectively removing the lesion 112 from within the lumen of the vessel 110 and opening the vessel. In cases where the balloon 100 is provided with cutting or scoring elements, the assembly 50 can be rotated as the balloon 100 is deployed, that is inflated or expanded, such that cutting or scoring elements on the balloon 100 can break away the material of the lesion 112, which can then be collected, if need be, by an aspiration catheter, as is known in the art.

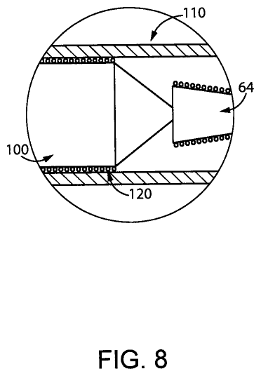

FIG. 8 is an expanded view of the portion of the assembly of FIG. 7 shown encircled, where it can be seen that the bioactive material layer 120 is pressed against the inner tissue membranes of the vessel 100 to deliver the bioactive material of the layer 120 directly to the vessel wall. This ensures the administration of bioactive agent directly to the vessel wall during the angioplasty procedure and, preferably, over short periods of time, which may be from one or more seconds to one or more minutes as described above.

Subsequent to the angioplasty procedure and administration of drug to the vessel 110, the balloon 100 can be deflated, in known manner, in order to retract the balloon from the vessel at the end of the procedure.

It will be appreciated that the drug coated medical balloon 100 is protected from external influences during the deployment of the balloon catheter assembly 50. The roll-sock 70 ensures that the drug or other bioactive agent 120 on the surface of the balloon cannot be scraped off the balloon or washed off by contact with the introducer sheath or blood flow during the deployment of the medical balloon. The reduction in wash off of drug or other bioactive agent minimises the loss of the drug or other bioactive agent into the blood stream and optimises the amount of drug or other bioactive agent that is in practice administered to the vessel wall.

The roll-sock 70 also ensures that the drug or other bioactive agent on the balloon can be administered accurately at the location where it is intended and also at the time when it is intended, particularly once it has been determined that the balloon is accurately positioned within the vessel. Only at that point, and once the physician is ready to deploy the balloon, does the roll-sock sleeve 70 need to be pulled back and it is only at this time that the drug or other bioactive agent comes into contact with the patient's blood flow. In practice, this can be an instant prior to inflation or expansion of the balloon and direct application of the drug or other bioactive agent to the vessel wall.

The hydrophilic or lubricous coating over at least the distal end of the assembly 50 makes it easier to pass the assembly through any calcification or stenosis.

The balloon catheter is advantageously free of any overlying stent. That is, there is no stent disposed over the balloon 100.

The apparatus 50 may also include a splitting mechanism for splitting the proximal end of the outer layer 116 of the roll-sock sleeve 70 at the proximal end of the assembly 50, that is when outside of the patient. There is preferably also provided a winding mechanism for rolling up the split portion of sleeve, in order to facilitate the handling of the roll sock sleeve 70 as it is withdrawn during deployment of the medical balloon 100. For this purpose, there could be provided a splitter and handle arrangement of the type disclosed in the applicant's earlier application U.S. Pat. No. 8,968,381, the disclosure of which is incorporated herein by reference.

In other embodiments, the roll-sock sleeve 70 need not extend all the way to the proximal end of the assembly 50 and could be replaced by another arrangement such as a wire or strip attached to a proximal end of the outer layer 116 of roll-sock sleeve 70 such that it is the wire or strip that is pulled to retract the roll-sock sleeve. In such case, the proximal end of the outer layer 116 can terminate at an intermediate position along the length of the balloon catheter 52, closer to the balloon 100. Any other mechanism could be used.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosure in the abstract accompanying this application is incorporated herein by reference.

1. A coated medical balloon assembly comprising:
a balloon catheter having a proximal end and a distal end, and a medical balloon mounted to the balloon catheter at or proximate the catheter distal end;
a bioactive material coating on at least the medical balloon;
a roll-sock sheath disposed over the medical balloon and at least a part of the balloon catheter, the roll-sock sheath comprising a first, inner, roll-sock layer disposed adjacent the medical balloon and a second, outer, roll-sock layer disposed over the first roll-sock layer, the roll-sock sheath including first and second roll-sock sheath ends and a fold line, the first and second roll-sock sheath ends being disposed at a proximal location relative to the fold line, the fold line being disposed adjacent the distal end of the balloon catheter, the first roll-sock sheath end being an end of the first roll-sock layer and the second roll-sock sheath end being an end of the second roll-sock layer;
a coating of hydrophilic or lubricous material on an outer surface of the second roll-sock layer and forming an outer layer of the coated medical balloon assembly;
a roll-sock sheath retraction mechanism coupled to the second roll-sock end and configured to retract the second roll-sock layer towards the proximal end of the catheter, whereby the first roll-sock layer progressively wraps over itself outwardly to the second roll-sock layer, thereby rolling back the roll-sock sheath and exposing the coated medical balloon for deployment.

2. An assembly according to claim 1, comprising a dilator tip at the distal end of the balloon catheter, the roll-sock sheath fold line being disposed adjacent the dilator tip when the roll-sock sheath is in a balloon overlying configuration, the coating of hydrophilic or lubricous material being disposed over the second roll-sock layer and the dilator tip.

3. An assembly according to claim 1, wherein the first roll-sock layer of the roll-sock sheath contacts the medical balloon when the roll-sock sheath is in a balloon overlying configuration.

4. An assembly according to claim 1, wherein the first end of the roll sock sheath is disposed at or adjacent a proximal end of the coated balloon.

5. An assembly according to claim 1, wherein the first end of the roll sock sheath is attached to the catheter at or adjacent the proximal end of the coated balloon.

6. An assembly according to claim 1, wherein the second end of the roll sock sheath is disposed at one of: (1) a location at or adjacent a proximal end of the catheter; and (2) an intermediate location of the catheter between its proximal and distal ends.

7. An assembly according to claim 1, comprising a roll-sock sheath winding mechanism operable to wind the second end of the roll-sock sheath during retraction thereof.

8. An assembly according to claim 7, comprising a roll-sock splitting device configured to split the second end of the roll-sock sheath longitudinally prior to winding on the winding mechanism, wherein the splitting mechanism splits the roll-sock sheath only at or adjacent the proximal end of the balloon catheter.

9. An assembly according to claim 1, comprising a low friction material disposed between the first and second layers of the roll-sock sheath.

10. An assembly according to claim 9, wherein the low friction material comprises one or more of: a material forming the roll-sock sheath, a layer of low friction compound between the first and second roll-sock layers of the roll-sock sheath, a liquid or gas disposed between the first and second roll-sock layers of the roll-sock sheath.

11. An assembly according to claim 10, wherein the layer of low friction compound is a layer of hydrophilic material.

12. An assembly according to claim 10, wherein the liquid or gas is disposed between the first and second roll-sock layers at the time of retraction of the roll-sock sheath, the apparatus comprising a device for locating such liquid or gas between the first and second roll-sock layers of roll-sock sheath at the time of retraction.

13. A method of deploying a coated medical balloon in a patient comprising:
providing a balloon catheter having a proximal end and a distal end, and a medical balloon mounted to the balloon catheter at or proximate the catheter distal end; a bioactive material coating on at least the medical balloon; a roll-sock sheath being disposed over the medical balloon and at least a part of the balloon catheter, the roll-sock sheath comprising a first, inner, roll-sock layer disposed adjacent the medical balloon and a second, outer, roll-sock layer disposed over the first roll-sock layer, the roll-sock sheath including first and second roll-sock sheath ends and a fold line, the first and second roll-sock sheath ends being disposed at a proximal location relative to the fold line, the fold line being disposed adjacent the distal end of the balloon catheter, the first roll-sock sheath end being an end of the first roll-sock layer and the second roll-sock sheath end being an end of the second roll-sock layer; a coating of hydrophilic or lubricous material on an outer surface of the second roll-sock layer and forming an outer layer of the coated medical balloon assembly;
feeding the assembly endoluminally through the vasculature of a patient, whereby the hydrophilic or lubricous coating eases the passage of the assembly through the vasculature;
retracting the second roll-sock layer, whereby the first roll-sock layer progressively wraps over itself outwardly to the second roll-sock layer to roll back the roll-sock sheath and expose the medical balloon for deployment, and whereby the hydrophilic or lubricous layer overlying the second roll-sock layer is retracted with the second roll-sock layer and such that contact between the hydrophilic or lubricous layer on the roll-sock sheath and the bioactive material layer on the balloon is avoided or minimised.

14. A method according to claim 13, wherein a dilator tip disposed at the distal end of the balloon is coated with the hydrophilic or lubricous material disposed over the second roll-sock layer, and wherein the step of feeding the assembly endoluminally through the vasculature of a patient is eased by the hydrophilic or lubricous coating over the roll-sock sheath and the dilator tip.

15. A method according to claim 13, wherein the first roll-sock layer of the roll-sock sheath contacts the medical balloon when the roll-sock sheath is in a balloon overlying configuration.

16. A method according to claim 13, comprising winding the second end of the roll-sock sheath during retraction thereof.

17. A method according to claim 16, comprising splitting the second end of the roll-sock sheath longitudinally prior to winding on the winding mechanism, wherein the splitting mechanism splits the roll-sock sheath only at or adjacent the proximal end of the balloon catheter.

18. A method according to claim 13, comprising disposing a low friction material between the first and second roll-sock layers of the roll-sock sheath.

19. A method according to claim 18, the low friction material comprises one or more of: a material forming the roll-sock sheath, a layer of low friction compound between the first and second roll-sock layers of the roll-sock sheath, a liquid or gas disposed between the first and second roll-sock layers of the roll-sock sheath.

20. A method according to claim 19, comprising the step of disposing the liquid or gas between the first and second roll-sock layers at the time of retraction of the roll-sock sheath.
